(19) United States
(12) Patent Application Publication
(10) Pub. No.: US 2026/0224598 A1
(43) Pub. Date: Aug. 6, 2026

(54) METHOD OF EXTRACTING A PSYCHOACTIVE COMPOUND FROM FUNGAL BIOMASS AND FORMULATIONS THEREOF

(71) Applicant: BLUETHERA BIOLOGICS INC., Calgary (CA)

(72) Inventors: Christianne STREET, Edmonton (CA); John SIMON, Edmonton (CA); Alon WOLFMAN, Calgary (CA)

(21) Appl. No.: 19/152,162

(22) PCT Filed: Nov. 24, 2024
(86) PCT No.: PCT/CA2023/051572

§ 371 Date: Jul. 30, 2025

Related U.S. Application Data
(60) Provisional application No. 63/442,146, filed on Jan. 31, 2023.
(60) Provisional application No. 63/454,650, filed on Mar. 25, 2023.
(60) Provisional application No. 63/454,649, filed on Mar. 25, 2023.
(60) Provisional application No. 63/454,648, filed on Mar. 25, 2023.
(60) Provisional application No. 63/454,647, filed on Mar. 25, 2023.
(60) Provisional application No. 63/495,161, filed on Apr. 10, 2023.

(51) Int. Cl. A61K 31/675; A61K 31/4045; A61K 31/437; A61K 47/22

(52) U.S. Cl. CPC A61K 31/675; A61K 31/4045; A61K 31/437; A61K 47/22; A61K 2236/15; A61K 2236/33; A61K 2236/37

(57) ABSTRACT

An aspect of the specification provides a method of extracting psychoactive compounds from fungal biomass by contacting the fungal biomass with pressurized carbon dioxide. The pressurized carbon dioxide is depressurized to obtain an extract containing at least one psychoactive compound, and the extract is collected into a collection vessel. The extract produced according to these methods may be used in the treatment of mental illness or migraines.

### CROSS-REFERENCE TO RELATED APPLICATIONS

This application claims the benefit of U.S. Provisional Patent Application No. 63/442,146 entitled “METHOD FOR PRODUCING PSYCHEDELIC-CONTAINING THERAPEUTICS FROM MUSHROOMS” filed Jan. 31, 2023, U.S. Provisional Patent Application No. 63/454,647 entitled “THERAPEUTIC USE OF PSILOCYBIN AND EFFECTIVE DOSAGE FORM DETERMINATION” filed Mar. 25, 2023, U.S. Provisional Patent Application No. 63/454,648 entitled “THERAPEUTIC USE OF PSILOCYBIN AND EFFECTIVE DOSAGE FORM DETERMINATION” filed Mar. 25, 2023, U.S. Provisional Patent Application No. 63/454,649 entitled “THERAPEUTIC USE OF PSILOCYBIN AND EFFECTIVE DOSAGE FORM DETERMINATION” filed Mar. 25, 2023, U.S. Provisional Patent Application No. 63/454,650 entitled “THERAPEUTIC USE OF PSILOCYBIN AND EFFECTIVE DOSAGE FORM DETERMINATION” filed Mar. 25, 2023, and U.S. Provisional Patent Application No. 63/495,161 entitled “PROCESS FOR THE CARBON DIOXIDE EXTRACTION OF PSYCHOACTIVE ALKALOIDS FROM MUSHROOMS” filed Apr. 10, 2023, the entire contents of which are incorporated herein by reference.

### FIELD

The present specification is directed to methods of extracting organic molecules from fungal biomass.

### BACKGROUND

Psychoactive compounds, such as psilocybin and psilocin, have remarkable pharmacological properties which are believed to be useful in the treatment of cluster headaches, depression, and other neurological disorders. These substances can be obtained from certain species of fungi and plants, however it is not commercially viable to extract psychoactive compounds from natural sources. Variability between batches and strains has stymied efforts to establish a repeatable dose. Furthermore, their reactivity renders psychoactive compounds more likely to degrade under acidic conditions and prone to oxidation when exposed to light.

### SUMMARY

The present disclosure addresses the limitations of the prior art by providing an improved method of extracting psychoactive compounds from fungal biomass. The method includes contacting the fungal biomass with pressurized carbon dioxide; depressurizing the pressurized carbon dioxide to obtain an extract containing at least one psychoactive compound and collecting the extract into a collection vessel. Extracts obtained according to the methods described herein can be manufactured with greater consistency and higher yields as compared with current methods. As a result, the present disclosure provides a commercially viable method of obtaining a standardized extract of psychoactive compounds from natural sources.

A further aspect of the specification provides a formulation comprising said extract.

It is an aspect of the present to provide an extract comprising a plurality of psychoactive compounds for the treatment of a mental illness selected from a group consisting of depression, anxiety, obsessive compulsive disorder (OCD), psychosis, post-traumatic stress disorder (PTSD), disordered eating, and addiction.

It is an aspect of the present to provide an extract comprising a plurality of psychoactive compounds for the treatment of migraines.

It is an aspect of the present to provide an extract comprising a plurality of psychoactive compounds for the treatment of cluster headaches.

It is an aspect of the present to provide an extract comprising a plurality of psychoactive compounds for the treatment of chronic pain.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a schematic diagram depicting a system for extracting a psychoactive compound from biomass.

FIG. 2 is a schematic diagram depicting another system for extracting a psychoactive compound from biomass.

FIG. 3 is a flowchart depicting a method of extracting a psychoactive compound from fungal biomass.

FIG. 4 is a flowchart depicting exemplary performance of the method of FIG. 3.

FIG. 5 is a flowchart depicting exemplary performance of the method of FIG. 3.

### DETAILED DESCRIPTION

### Acronyms

The following acronyms are used herein:

| | |
|---|---|
| ASTM | American Society for Testing and Materials |
| BHA | butylated hydroxyanisole |
| BHT | butylated hydroxytoluene |
| CO₂ | carbon dioxide |
| DMSO | dimethyl sulfoxide |
| GMP | good manufacturing practices |
| HPLC | high pressure liquid chromatography |
| K | Kelvin |
| MPa | megapascal |
| NMR | nuclear magnetic resonance |
| OCD | obsessive compulsive disorder |
| PTSD | post-traumatic stress disorder |
| QA/QC | quality assurance and quality control |
| TBHQ | tertiary butylhydroquinone |
| TLC | thin layer chromatography |
| USP | United States Phamaceopeia |

### Definitions

“About” herein refers to a range of +20% of the numerical value that follows. In one embodiment, the term “about” refers to a range of +10% of the numerical value that follows. In one embodiment, the term “about” refers to a range of +5% of the numerical value that follows.

“Atmospheric pressure” herein refers to about 101.325 kPa.

“Atmospheric conditions” herein refers to air having the same or similar composition as the Earth's atmosphere. In particular, atmospheric air comprises approximately 21% oxygen.

“Basidiocarp” herein refers to the fruiting body of a fungus, particularly a basidiomycete.

“Biomass” herein refers to organic matter, in whole or in part, which is derived from one or more fungi.

“Extract” herein refers to a solution or mixture obtained by extracting one or more compounds from biomass.

“Low-oxygen conditions” herein refers to air having a composition of oxygen that is lower than the composition of oxygen in the Earth's atmosphere.

“Purging” herein refers to the process of flowing nitrogen gas, through an enclosure or system to remove unwanted gases such as oxygen and water vapour.

“Psilocybin” herein refers to a psychoactive alkaloid having the general formula C₁₂H₁₇N₂O₄P, also known as “3-[2-(Dimethylamino)ethyl]-1H-indol-4-yl dihydrogen phosphate”.

“Psilocin” herein refers toa psychoactive alkaloid having the general formula C₁₂H₁₆N₂O, also known as “3-[2-(Dimethylamino)ethyl]-1H-indol-4-ol”.

“Psilocybe” herein refers to a genus of gilled mushrooms in the family Hymenogastraceae which is known for producing psychoactive compounds. Psilocybe includes Psilocybe cubensis, Psilocybe subcubensis, Psilocybe syanescens, Psilocybe montana, Psilocybe semilanceata, and other species.

“Psychoactive compound” herein refers to a chemical compound that produces a change in brain function resulting in alterations of mood, cognition, or behavior, for example.

“Sclerotium” (plural: “sclerotia”) herein refers to a compact portion of a fungal root or “mycelium” which contains store nutrients for the fungus and may appear dark. In psychoactive mushrooms, the sclerotium contains psychoactive compounds and is sometimes referred to informally as a “magic truffle”.

“Supercritical solvent” refers to a substance that that is subjected to a temperature and pressure above its critical point. A supercritical solvent exhibits properties of both liquids and gases.

### System and Methods

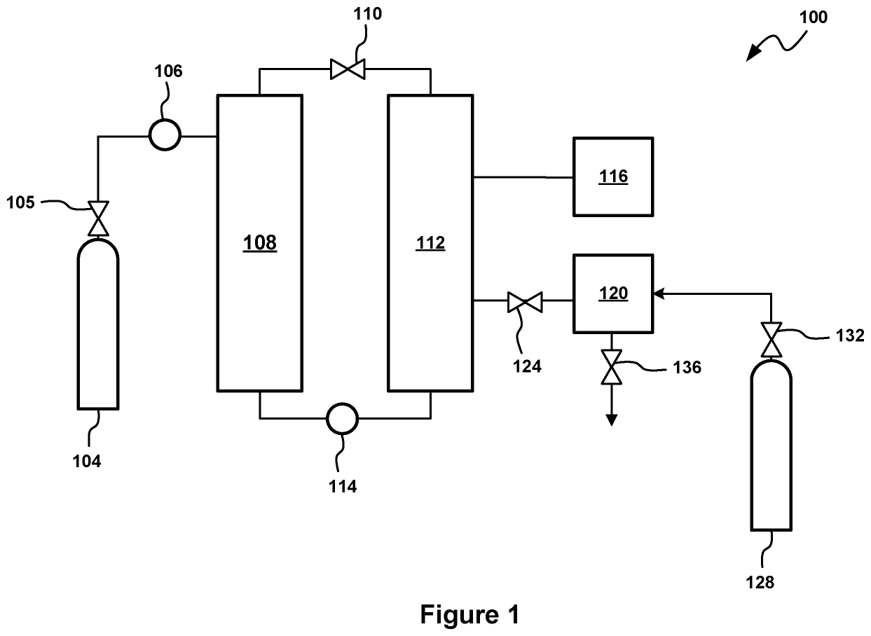

FIG. 1 shows a system 100 for extracting psychoactive compounds from biomass. System 100 includes a gas source 104, an extraction vessel 108, a separator 112, a waste receptacle 116, and a collection vessel 120.

The gas source 104 includes any suitable source of carbon dioxide (CO₂). In some examples, the gas source 104 comprises a cylinder of compressed carbon dioxide gas. The gas source 104 may include a first valve 105 for controlling the release of carbon dioxide (CO₂) from the cylinder.

The gas source 104 is connected to the extraction vessel 108 by a conduit for conveying the carbon dioxide from the gas source 104 to the extraction vessel 108. The conduit is further connected to a pump 106 for pressurizing the carbon dioxide. Although not shown, the system 100 may further include a heater for heating the carbon dioxide.

The extraction vessel 108 comprises a pressurized container for receiving the CO₂ and biomass. One example of a commercially available extraction vessel is Superfast 2×9 from Thar Process (Pittsburgh, PA).

The extraction vessel 108 is connected by a conduit to the separator 112 which is configured to separate the CO₂ from the extract. The separator 112 may comprise a commercially available pressure vessel such as such as the Superfast™ 2×9 from Thar Process (Pittsburgh, PA). A second valve 110 is positioned between the extraction vessel 108 and the separator 112 to control the flow of CO₂ from the extraction vessel 108 to the separator 112. The second valve 110 may be a needle valve or a flow control valve, however the second valve 110 is not particularly limited. Generally, the second valve 110 imposes friction on the substances flowing between the extraction vessel 108 and the separator 112. In the example shown, the second valve 110 is spaced between the extraction vessel 108 and the separator 112, however, the second valve 110 is not particularly limited. In other examples, the second valve 110 is included at an outlet of the separator 112. The separator 112 may further include a back pressure regulator for controlling the air pressure in the separator 112. The separator 112 may further include a filter for separating the biomass from the extract. CO₂ may be recycled from the separator 112 to the extraction vessel 108 via a conduit and a pump 114. In the example shown, the pump 114 is spaced between the separator 112 and the extraction vessel 108, but the pump 114 is not particularly limited. The extraction vessel 108 may further include an exhaust port (not shown) for venting the CO₂ to the atmosphere or into a CO₂ collection tank when the extraction is complete.

The separator 112 may be connected to the waste receptacle 116 for receiving waste from the separator 112.

The separator 112 is further connected to the collection vessel 120 for collecting the extract. The collection vessel 120 may comprise any suitable container for receiving the extract. The collection vessel 120 may be hermetically sealed to the separator 112. In the example shown in FIG. 1, the collection vessel 120 is configured to be purged with a gas, although the system 100 is not particularly limited. In this example, a third valve 124 connects the collection vessel 120 to the separator 112 for controlling the flow of extract into the collection vessel 120. In the example shown, the third valve 124 is spaced between the separator 112 and the collection vessel 120, but the third valve 124 is not particularly limited. The collection vessel 120 is further connected to a gas source 128 for supplying a purging gas to the collection vessel 120. The purging gas is generally an inert gas which may include, but is not limited to, nitrogen gas (N₂), carbon dioxide (CO₂), argon, and helium. In some examples, the gas source 128 comprises a cylinder of compressed purging gas. The gas source 128 may include a fourth valve 132 for controlling the release of the purging gas from the cylinder. The collection vessel 120 may further include an exhaust valve 136 for releasing the purging gas through an exhaust port.

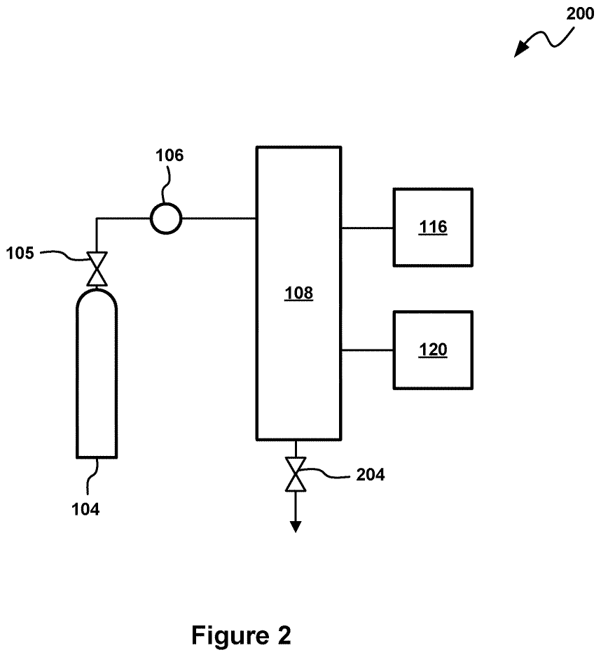

FIG. 2 is a schematic diagram depicting another system 200 for extracting a psychoactive compound from biomass. System 200 is a variant of system 100. In system 200, the separator 112 is omitted and the extraction vessel 108 is instead connected to the waste receptacle 116 and collection vessel 120. In the system 200, the extraction vessel 108 includes an exhaust valve 204 for controlling the release of CO₂ through an exhaust port. The exhaust valve 204 may be suitable for quickly opening and closing the exhaust port. In specific non-limiting examples, the exhaust valve 204 is a low friction, on/off valve. The exhaust port may have a diameter that is large enough to rapidly release the pressure in extraction vessel 108. Generally, the system 200 is suitable for CO₂ explosion because the air pressure in the extraction vessel 108 can be decreased within a short period of time. CO₂ explosion may warp or break the cell walls of the fungal biomass, improving CO₂ penetration which in turn enhances the extraction efficiency.

In the example shown in FIG. 2, the collection vessel 120 is not configured for purging, however system 200 is not particularly limited. In other examples, system 200 includes the third and fourth valves 124, 132, the exhaust valve 136, and the gas source 128. It should be understood the methods described herein may be performed on either the supercritical CO₂ system 100 or the CO₂ explosion system 200.

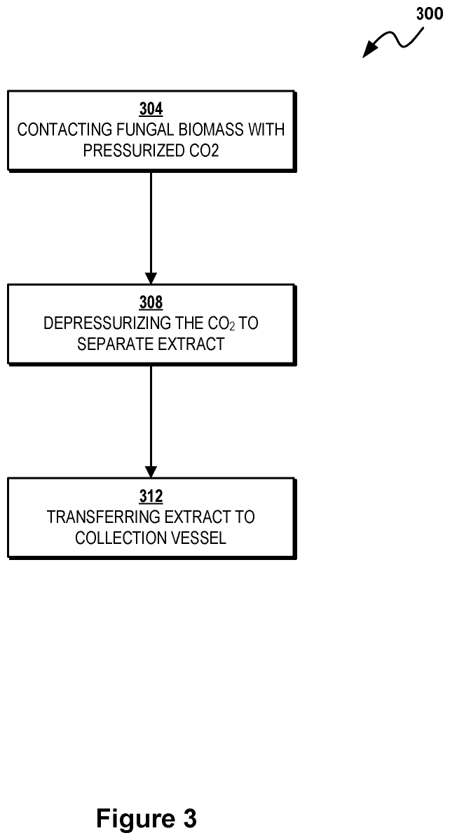

FIG. 3 shows a method 300 for extracting psychoactive compounds from biomass. The method 300 may be performed by the system 100 or system 200.

At block 304, fungal biomass is contacted with pressurized carbon dioxide (CO₂). In the system 100 and system 200, block 304 is performed in the extraction vessel 108 which receives the fungal biomass and the pressurized CO₂.

The fungal biomass includes at least a portion of a fungus that comprises at least one psychoactive compound (generically referred to herein as “the psychoactive compound” or collectively as “the psychoactive compounds”). In some examples, the fungal biomass includes the whole fungus, and in other examples, the fungal biomass includes a portion of the fungus. In examples where the fungal biomass includes a portion of the fungus, the fungal biomass comprises any suitable portion of the fungus including, but not limited to, the fruiting body (also known as the “basidiocarp”) or the sclerotia. In some examples, the fungal biomass comprises for the cap and the stem of the fruiting body, but in other examples, the fungal biomass comprises only the cap of the fruiting body.

The fungal biomass may include, but is not limited to, Copelandia sp., Galerina sp., Gymnopilus sp., Incobye sp., Panaeolus sp., Pholiotina sp., Pluteus sp., and Psilocybe sp. In specific, non-limiting examples, the fungal biomass includes Conocybe keuhneriana, Conocybe siligineoides, Conocybe velutipes, Galerina steglichii, Gymnopilus luteofolius, Gymnopilus aeruginosas, Gymnopilus braendlei, Gymnopilus cyanopalmicola, Gymnopilus dilepis, Gymnopilus dunensis, Gymnopilus intermedius, Gymnopilus lateritius, Gymnopilus liquiritiae, Gymnopilus luteofolius, Gymnopilus luteoviridis, Gymnopilus luteus, Gymnopilus palmicola, Gymnopilus purpuratus, Gymnopilus subpurpuratus, Gymnopilus spectabilis, Gymnopilus subspectabilis, Gymnopilus validipes, Gymnopilus viridans, Inocybe aeruginascens, Inocybe aeruginascens, Inocybe caerulata, Inocybe coelestium, Inocybe corydalina, Inocybe haemacta, Inocybe tricolor, Panaeolus cinctulus, Panaeolus affinis, Panaeolus africanus, Panaeolus axfordii, Panaeolus bisporus, Panaeolus cambodginiensis, Panaeolus chlorocystis, Panaeolus cinctulus, Panaeolus cyanescens, Panaeolus fimicola, Panaeolus lentisporus, Panaeolus microsporus, Panaeolus moellerianus, Panaeolus olivaceus, Panaeolus rubricaulis, Panaeolus tirunelveliensis, Panaeolus tropicalis, Panaeolus venezolanus, Pholiotina cyanopus, Pholiotina smithii, Pluteus americanus, Pluteus albostipitatus, Pluteus americanus, Pluteus cyanopus, Pluteus ephebus, Pluteus glaucus, Pluteus glaucotinctus, Pluteus nigroviridis, Pluteus phaeocyanopus, Pluteus salicinus, Pluteus saupei, Pluteus velutinornatus, Pluteus villosus, Psilocybe atlantis, Psilocybe acutipilea, Psilocybe allenii, Psilocybe alutacea, Psilocybe angulospora, Psilocybe antioquiensis, Psilocybe araucariicola, Psilocybe atlantis, Psilocybe aquamarina, Psilocybe argentipes, Psilocybe armandii, Psilocybe aucklandiae, Psilocybe australiana, Psilocybe aztecorum, Psilocybe azurescens, Psilocybe baeocystis, Psilocybe banderillensis, Psilocybe bohemica, Psilocybe brasiliensis, Psilocybe brunneocystidiata, Psilocybe cubensis, Psilocybe caeruleoannulata, Psilocybe caerulescens, Psilocybe caerulipes, Psilocybe callosa, Psilocybe carbonaria, Psilocybe chuxiongensis, Psilocybe collybioides, Psilocybe columbiana, Psilocybe congolensis, Psilocybe cordispora, Psilocybe cubensis, Psilocybe cyanescens, Psilocybe cyanofibrillosa, Psilocybe dumontii, Psilocybe egonii Guzmán, Psilocybe eximia, Psilocybe fagicola, Psilocybe farinacea, Psilocybe fimetaria, Psilocybe fuliginosa, Psilocybe furtadoana, Psilocybe tampanensis, Psilocybe galindoi, Psilocybe gallaeciae, Psilocybe graveolens, Psilocybe guatapensis, Psilocybe guilartensis, Psilocybe heimii, Psilocybe herrerae, Psilocybe hispanica, Psilocybe hoogshagenii, Psilocybe hopii, Psilocybe inconspicua, Psilocybe indica, Psilocybe isabelae, Psilocybe jacobsii, Psilocybe jaliscana, Psilocybe kumaenorum, Psilocybe laurae, Psilocybe lazoi, Psilocybe liniformans, Psilocybe mexicana, Psilocybe mairei, Psilocybe makarorae, Psilocybe mammillata, Psilocybe medullosa, Psilocybe meridensis, Psilocybe mescaleroensis, Psilocybe mexicana, Psilocybe moseri, Psilocybe muliercula, Psilocybe naematoliformis, Psilocybe natalensis, Psilocybe natarajanii, Psilocybe neorhombispora, Psilocybe neoxalapensis, Psilocybe ovoideocystidiata, Psilocybe papuana, Psilocybe paulensis, Psilocybe pelliculosa, Psilocybe pintonii, Psilocybe pleurocystidiosa, Psilocybe plutonia, Psilocybe portoricensis, Psilocybe pseudoaztecorum, Psilocybe puberula, Psilocybe quebecensis, Psilocybe rickii, Psilocybe rostrata, Psilocybe rzedowskii, Psilocybe samuiensis, Psilocybe schultesii, Psilocybe semilanceata, Psilocybe septentrionalis, Psilocybe serbica, Psilocybe sierrae, Psilocybe silvatica, Psilocybe singeri, Psilocybe strictipes, Psilocybe stuntzii, Psilocybe subacutipilea, Psilocybe subaeruginascens, Psilocybe subaeruginosa, Psilocybe subbrunneocystidiata, Psilocybe subcaerulipes, Psilocybe subcubensis, Psilocybe subpsilocybioides, Psilocybe subtropicalis, Psilocybe tampanensis, Psilocybe tasmaniana, Psilocybe thaiaerugineomaculans, Psilocybe thaicordispora, Psilocybe thaiduplicatocystidiata, Psilocybe uruguayensis, Psilocybe uxpanapensis, Psilocybe venenata, Psilocybe wassoniorum, Psilocybe wayanadensis, Psilocybe weilii, Psilocybe weldenii, Psilocybe weraroa, Psilocybe xalapensis, Psilocybe yungensis, Psilocybe zapotecoantillarum, Psilocybe zapotecocaribaea, Psilocybe zapotecorum, or a combination thereof. In examples described herein, the fungal biomass comprises Psilocybe cubensis, however the fungal biomass is not particularly limited.

Advantageously, the fungal biomass comprises immature mushrooms because immature mushrooms have higher concentrations of psychoactive compounds such as psilocin as compared to mature mushrooms.

In system 100 and system 200, the fungal biomass is contacted with the pressurized CO₂ in the extraction vessel 108.

The system 100, 200 controls the pressure of the extraction vessel 108 to exceed atmospheric pressure. In specific, non-limiting examples, the system 100, 200 includes the pump 106 which controls the pressure of the extraction vessel 108. In the example shown, the pump 106 is spaced between the gas source 104 and the extraction vessel 108, but the pump 106 is not particularly limited.

In system 100, the pump 106 controls the air pressure in the extraction vessel 108 to exceed about 7.4 MPa (megapascals). In some examples, the pump 106 controls the pressure of the extraction vessel 108 between 7.4 MPa and 35 MPa. The system 100 may further include a heater for controlling the temperature of the extraction vessel 108 to exceed about 304.13 K (Kelvin). In some examples, the heater controls the temperature of the extraction vessel 108 between about 304.1 K to about 360 K. At pressure above about 7.4 MPa and temperatures above about 304.13 K, CO₂ is supercritical.

In system 200, the pump 106 controls the air pressure in the extraction vessel 108 to exceed 4 MPa. In specific non-limiting examples, the pump 106 controls the pressure of the extraction vessel 108 between 4 MPa and 7.4 MPa. The system 200 may further include a heater for controlling the temperature of the extraction vessel 108.

As a solvent, CO₂ is advantageous because it is non-flammable and leaves no residue. Furthermore, supercritical CO₂ has no surface tension, allowing it to penetrate the fungal biomass and increase extraction yields.

The pump 106 may force the CO₂ through the extraction vessel 108 at a flow rate of about 1 to about 10 kg (kilograms) per hour.

The contact time between the fungal biomass and the pressurized CO₂ may last between 1 minute and 24 hours. In some examples, the contact time is about 1 minute. In some examples, the contact time is between 1 and 4 hours. In specific non-limiting examples, the contact time is about 3 hours. In other examples, the contact time is between 8 and 24 hours.

Block 304 may further include contacting the fungal biomass with a co-solvent. The co-solvent may be added to the extraction vessel 108. The co-solvent may be a polar, non-toxic solvent. The co-solvent may include but is not limited to, water, acetone, hexane, ethylene glycol, pyridine, acetic acid, chloroform, ethyl acetate, dimethyl sulfoxide (DMSO), ethanol (ethyl alcohol), methanol (methyl alcohol), or a combination thereof. In specific non-limiting examples, the concentration of the co-solvent is between 5% and 10% (v/v). Generally, the co-solvent is selected to alter the polarity of the pressurized CO₂ to improve the overall extraction of polar molecules. Since CO₂ is mostly non-polar, the co-solvent is typically a polar compound.

Contacting the fungal biomass with pressurized CO₂ at block 304 causes an extract of the fungal biomass to be dissolved in the pressurized CO₂. Collectively, the extract and the pressurized CO₂ will be referred to herein as the “extraction mixture”. The extract includes at least one psychoactive compound. In some examples, the extract includes a plurality of psychoactive compounds.

The psychoactive compound includes one or more substances that affects mental processes in a subject and may include a hallucinogen, depressant, or stimulant. In particular, non-limiting examples, the psychoactive compound includes an alkaloid. The psychoactive compound may include but is not limited to psilocin, norpsilocin, psilocybin, aeruginascin, baeocystin, a β-carboline (including but not limited to cordysinin C, cordysinin D, harmane, harmol, norharmane, and perlolyrine), bufotenin, norbaeocystin, norpsilocin, O-acetylpsilocin, or a combination thereof. The psychoactive compound may include any suitable number of psychoactive compounds.

At block 308, the extraction mixture is depressurized.

In system 100, block 308 comprises conveying the extraction mixture to the separator 112. In system 100, block 308 is performed by a conduit which conveys the extraction mixture from the extraction vessel 108 to the separator 112. As part of block 308, the second valve 110 may be opened, allowing the supercritical carbon dioxide to flow from the extraction vessel 108 to the separator 112. In some examples, the flow of carbon dioxide from the extraction vessel 108 to the separator 112 is aided by a pump connected to the conduit. As part of block 308, the extract may be separated from exhausted biomass material when the extraction mixture is conveyed from the extraction vessel 108. The extract is conveyed by the carbon dioxide into the separator 112 and the exhausted biomass material remains in the extraction vessel 108. Since the air pressure in the separator 112 is below the supercritical point for carbon dioxide, the extraction mixture is depressurized when the extraction mixture passes through the second valve 110. The depressurization occurs rapidly, and in some examples, less than one second. The depressurization causes the extract to separate from the carbon dioxide in the extraction mixture.

The pressure in the separator 112 may be controlled by a pressure regulator and particularly a back pressure regulator. As part of block 308, some of the CO₂ may be released from the separator 112 either by channeling the CO₂ into the waste receptacle 116, returning the CO₂ to the extraction vessel 108 via the pump 114 for subsequent use, or releasing the CO₂ into the atmosphere via an exhaust valve. In examples where the CO₂ is returned to the extraction vessel 108, the pump 114 restores the pressure of the CO₂.

In examples where the method 300 is performed on the CO₂ explosion system 200, block 308 comprises depressurizing the extraction vessel 108. In these examples, block 308 is performed by exhaust valve 204 which releases some of the CO₂ from the extraction vessel 108 so that the air pressure in the extraction vessel 108 decreases. In system 200, the depressurization at block 308 can be performed rapidly. In some examples, depressurization occurs in about two minutes. In other examples, depressurization occurs in about one minute. In further examples, depressurization occurs in about thirty seconds. In yet further examples, depressurization occurs in less than 15 seconds.

It should be understood that blocks 304 to 308 may be repeated on the fungal biomass. After depressurization at block 308, the extraction vessel 108 may be repressurized to continue extraction.

At block 312, the extract is conveyed into the collection vessel 120.

In one example, the extract may be removed from the separator 112 by applying pressure to the separator, causing the extract to spray into the collection vessel 120. In another example, the separator 112 is rinsed with a solution to remove the extract into the collection vessel 120.

The collection vessel 120 may contain a preservative to inhibit oxidation of the psychoactive compound. The preservative includes, but is not limited to, α-tocopherol (alpha-tocopherol or vitamin E), butylated hydroxyanisole (BHA), butylated hydroxytoluene (BHT), and tertiary butylhydroquinone (TBHQ), the purging gas, or a combination thereof. In examples where the preservative comprises the purging gas, the collection vessel 120 is purged with the purging gas before collecting the extract. Purging the collection vessel includes venting the purging gas from the gas source 128 into the collection vessel 120 and out through the exhaust valve 136. The purging gas may be circulated through the collection vessel 120 for a pre-determined amount of time in order to displace any oxygen from the collection vessel 120. In specific non-limiting examples, the collection vessel 120 is purged for a period of 1 to 10 minutes. The duration of time may depend on the size of the collection vessel 120 and the rate at which the gas is flowing through the collection vessel 120. Then, the exhaust valve 136 is closed and the fourth valve 132 is closed. To collect the extract into the purged collection vessel 120, both the third valve 124 and the exhaust valve 136 are opened allowing the extract and the CO₂ in the separator 112 to flow into the collection vessel. The flow of CO₂ and extract into the collection vessel 120 displaces at least a portion of the purging gas through the exhaust valve 136. Once the extract has been collected, the third valves 124 and the exhaust 136 may be closed to seal the collection vessel 120.

In view of the above, it will now be apparent that variant, combinations, and subsets of the foregoing embodiments are contemplated.

In some examples, the extraction with CO₂ may include contacting the fungal biomass with CO₂ in a flow-through scheme. In the flow-through scheme, the fungal biomass is input to the extraction vessel as a layer, then the CO₂ is pumped through the layer of biomass.

In other examples, the extraction with CO₂ may include contacting the fungal biomass with CO₂ in a counter-current flow scheme. In the counter-current flow scheme, the fungal biomass is pumped under pressure to the top of the commercially available pressure vessel to flow downward against the upward flow of the CO₂.

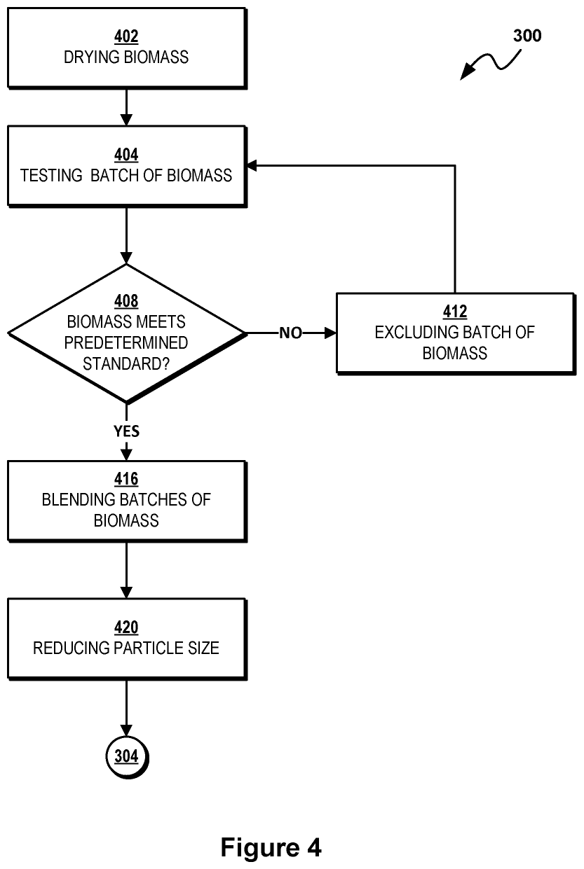

According to another variation, the fungal biomass undergoes processing steps prior to extraction. FIG. 4 shows exemplary performance of method 300 according to another embodiment. The blocks shown in FIG. 4 may be performed in any order and one or more blocks may be omitted. Generally, the blocks shown in FIG. 4 are performed prior to contacting the fungal biomass with the pressurized CO₂ at block 304.

At block 402, the fungal biomass is dried. The fungal biomass may be dried using any suitable procedure including but not limited to vacuum drying, rotary evaporating, freeze drying, hot-air drying, microwave with hot air, infrared with hot air, and combinations thereof. The dried fungal biomass may have a moisture content between 0% and 10 ±2%. In specific embodiments, the dried fungal biomass has a moisture content between 0% and 5 ±1%. In further embodiments, the dried biomass has a moisture content between 0% and 2 ±0.4%. In further embodiments, the dried fungal biomass has a moisture content between about 0% and 1 ±0.2%. In specific non-limiting embodiments, the dried fungal biomass has a moisture content of 2 ±0.4%. In specific non-limiting embodiments, the dried biomass has a moisture content of 1 ±0.2%.

It should be understood that block 402 may be omitted and that in other examples, the fungal biomass is wet when it contacts the pressurized CO₂ in the extraction vessel 108.

To ensure consistency in the composition of the extract, a batch of the fungal biomass may be tested at block 404. Testing includes but is not limited to testing the batch for the presence or quantity of a characteristic. The characteristic may include, but is not limited to, alkaloids, polysaccharides proteins, fats, minerals, glycosides, terpenoids, folates, tocopherols, flavonoids, phenolics, volatile oils, ascorbic acid, lectins, enzymes, organic acids, appearance, moisture, contaminants, undesired materials, or a combination thereof. Contaminants may include, but are not limited to bacteria, yeast, mold, aflatoxins, heavy metals, or combinations thereof. Undesired materials may include, but are not limited to, chitin, beta glucan or other compounds known to cause side effects upon ingestion of the psychoactive compound, or combinations thereof. Typically, the testing procedure is selected according to the characteristic. The testing procedure includes but is not limited to high pressure liquid chromatograph (HPLC), thin layer chromatography (TLC), nuclear magnetic resonance (NMR) spectroscopy, mass spectrometry, solid phase extraction, electrophoresis, western blotting, bacteria culture, and combinations thereof. In certain examples, the characteristic may include the appearance of the batch and the testing procedure includes visual examination of the batch for mold, rot, slime, or other spoilage, color, or a combination thereof.

At block 408, the characteristic identified or quantified at block 404 is compared to a pre-determined standard. The pre-determined standard may be selected according to said characteristic. In some examples, the pre-determined standard comprises a minimum or maximum value for the characteristic. In other examples, the pre-determined standard comprises a range of acceptable values for the characteristic.

If the batch of biomass fails to meet the pre-determined standard, the batch is excluded at block 412 and the method 300 returns to block 404. Block 404 is repeated for a new batch of biomass. If the batch of biomass meets the pre-determined standard, the method 300 proceeds to block 416.

Blocks 404 to 412 may be repeated for two or more batches of biomass until a desired cumulative quantity of biomass is reached.

Block 316 comprises blending two or more batches of biomass. Block 416 may be performed manually or with a machine mixer. Generally, blending the batches of biomass improves uniformity of the extract. Blending can also improve control over the composition of the extract. For example, blending a specific mass of high potency mushrooms with a specific mass of low potency mushrooms can result in a combined biomass having mid-potency.

Block 420 comprises reducing the average particle diameter of the fungal biomass. Block 420 may be performed with any suitable device including but not limited to a mortar and pestle, a food processor, a blender, and combinations thereof. In some embodiments, the average particle diameter of the fungal biomass is between 200 ±40 μm and 2000 ±400 μm after the performance of block 420. In some embodiments, the average particle diameter of the biomass is between 400 ±80 μm and 1200 ±240 μm after the performance of block 420. In some embodiments, the average particle diameter of the fungal biomass is between 600 ±100 μm and 800 ±160 μm after the performance of block 420. As part of block 420, the fungal biomass may be passed through one or more sieve plates to control the average particle diameter of the fungal biomass.

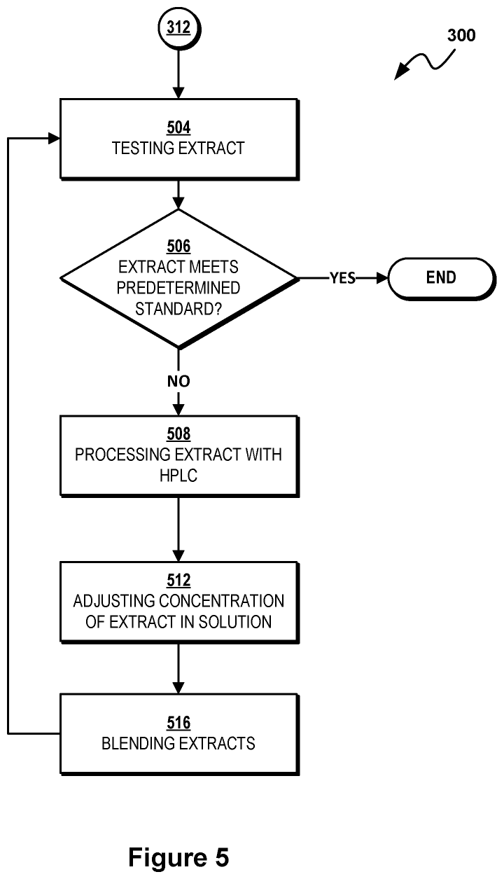

After the extract is collected at block 312, further processing steps may be performed. FIG. 5 shows exemplary performance of method 300 according to another embodiment. The blocks shown in FIG. 5 may be performed in any order and one or more blocks may be omitted.

Block 504 comprises testing the extract. As part of block 504, the extract may be dried to remove solvents. Any suitable drying technique may be used including vacuum drying, rotary evaporating, freeze drying, hot-air drying, microwave with hot air, infrared with hot air, and combinations thereof. Drying the extract results in a dried, powdered extract.

Testing includes evaluating the extract for the presence or quantity of a substance including but not limited to, alkaloids, polysaccharides proteins, fats, minerals, glycosides, terpenoids, folates, tocopherols, flavonoids, phenolics, volatile oils, ascorbic acid, lectins, enzymes, organic acids, appearance, moisture, contaminants, undesired materials, or a combination thereof. Contaminants may include, but are not limited to bacteria, yeast, mold, aflatoxins, heavy metals, or combinations thereof. Undesired materials may include, but are not limited to, chitin, beta glucan or other compounds known to cause side effects upon ingestion of the psychoactive compound, or combinations thereof. Typically, the testing procedure is selected according to the substance being tested. The testing procedure includes but is not limited to mass balance, thin layer chromatography (TLC), high pressure liquid chromatograph (HPLC), nuclear magnetic resonance (NMR) spectroscopy, mass spectrometry, solid phase extraction, electrophoresis, western blotting, bacteria culture, or combinations thereof.

In examples where block 504 comprises mass balance, the batch of biomass is tested at block 404 for the quantity of the psychoactive compound, and after the extraction is collected at block 312, the extract is tested for the quantity of the psychoactive compound.

At block 506, the substance identified or quantified at block 504 is compared to a pre-determined standard. The pre-determined standard may be selected according to said substance. In some examples, the pre-determined standard comprises a minimum or maximum value for the substance. In other examples, the pre-determined standard comprises a range of acceptable values for the substance.

Block 506 may include comparing the substance identified or quantified at block 504 with the composition of the biomass batch. In specific examples, the mass of the psychoactive compound in the biomass batch is compared to the mass of the psychoactive compound in the exhausted material and the mass of the psychoactive compound in the extract. This comparison can be used to verify the results of the testing at block 504.

If the extract fails to meet the pre-determined standard, the extract may be discarded or subject to further processing in order to adjust the characteristic of the extract. Blocks 508, 512, and 516 describe non-limiting examples of steps that may be taken to adjust the characteristic of the extract.

Block 508 comprises processing the extract with high pressure liquid chromatography (HPLC). HPLC may be used to isolate, remove, or concentrate any number of compounds in the extract. In some examples, HPLC is used to remove a compound such as a contaminant or undesired material. In other examples, HPLC is used to concentrate the psychoactive compounds. In further examples, HPLC is used to isolate the psychoactive compound. As part of block 508, a solvent may be added to the extract.

Block 512 comprises adjusting the concentration of the extract in solution. Block 512 may be performed by diluting the extract or evaporating the solvent from the extract. In some examples, the extract is diluted by addition of a solvent. Suitable solvent includes, but are not limited to water, acetone, hexane, ethylene glycol, pyridine, acetic acid, chloroform, ethyl acetate, dimethyl sulfoxide (DMSO), ethanol, methanol, or a combination thereof. In other examples, the extract is concentrated by evaporating the extract. In further examples, the extract is concentrated by HPLC.

Block 516 comprises blending two or more extracts obtained from repeated performance of method 300. Block 516 may be performed by manually blending or with a mixing machine.

The extracts obtained from repeated performance of method 300 may differ in composition. If it is determined at block 506 that the composition of a first extract does not meet the predetermined standard, the first extract may be combined with a second extract at block 516. In a specific non-limiting example, the concentration of the psychoactive compound in a first extract is below the pre-determined standard and the concentration of the psychoactive compound in a second extract exceeds the pre-determined standard. The first and second extracts may be blended at block 516 to produce an extract which meets the pre-determined standard.

In some examples, only a portion of the first extract is blended with the second extract. In other examples, only a portion of the second extract is blended with the first extract. In further examples, a portion of the first extract is blended with a portion of the second extract.

After adjusting the composition of the extract in blocks 508, 512 and 516, the method returns to block 504 for further testing. If it is determined that the extract meets the predetermined standard, the method 300 may end.

The extract comprises at least one psychoactive compound. In some examples, the extract comprises a plurality of psychoactive compounds from the fungal biomass. Advantageously, the extract comprises a plurality of psychoactive compounds that work synergistically together to elicit psychoactive effects in a subject.

The extract may be used in the manufacture of a medicament for the treatment of mental health disorders including but not limited to depression, anxiety, obsessive compulsive disorder (OCD), psychosis, post-traumatic stress disorder (PTSD), eating disorders, and addiction.

The extract may be used in the manufacture of a medicament for the treatment of migraines.

The extract may be used in the manufacture of a medicament for the treatment of cluster headaches.

The extract may be used in the manufacture of a medicament for the treatment of chronic pain.

It is a further aspect of the disclosure to provide a formulation comprising the extract obtained by the method 300. The formulation comprises a pharmaceutically acceptable amount of the extract. In certain examples, the formulation comprises an oral tablet, a liquid suspension, a capsule, an injection, a suppository, a topical cream or ointment, a nasal spray, an inhalable solution, an eye drop, transdermal patch, or the like.

The formulation may further comprise a solvent. Suitable solvents include but are not limited to water, ethanol (ethyl alcohol), acetone, benzyl alcohol, 1,3-butylene glycol, carbon dioxide, castor oil, citric acid esters of mono- and di-glycerides, ethyl acetate, ethyl alcohol denatured with methanol, glycerol (glycerin), glyceryl diacetate, glyceryl triacetate (triacetin), glyceryl tributyrate (tributyrin), hexane, isopropyl alcohol (isopropanol), methyl alcohol (methanol), methyl ethyl ketone (2-butanone), methylene chloride (dichloromethane), monoglycerides and diglycerides, monoglyceride citrate, 2-nitropropane, 1,2-propylene glycol (1,2-propanediol), propylene glycol mono-esters and diesters of fat-forming fatty acids, triethyl citrate, and combinations thereof.

The formulation may further include a buffering agent. The buffering agent includes, but is not limited to, acetic acid, ammonium aluminum sulphate, ammonium bicarbonate, ammonium carbonate, ammonium citrate, dibasic, ammonium citrate, monobasic, ammonium hydroxide, ammonium phosphate, dibasic, ammonium phosphate, monobasic, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, dibasic, calcium phosphate, monobasic, calcium phosphate, tribasic, calcium sulphate, carbon dioxide, citric acid, cream of tartar, fumaric acid, gluconic acid, glucono-delta-lactone, hydrochloric acid, lactic acid, magnesium carbonate, magnesium citrate, magnesium fumarate, magnesium hydroxide, magnesium oxide, magnesium phosphate, magnesium sulphate, malic acid, manganese sulphate, metatartaric acid, phosphoric acid, potassium acid tartrate, potassium aluminum sulphate, potassium bicarbonate, potassium carbonate, potassium chloride, potassium citrate, potassium fumarate, potassium hydroxide, potassium lactate, potassium phosphate, dibasic, potassium phosphate, tribasic, potassium pyrophosphate, tetrabasic, potassium sulphate, potassium tartrate, potassium tripolyphosphate, sodium acetate, sodium acid pyrophosphate, sodium acid tartrate, sodium aluminum phosphate, sodium aluminum sulphate, sodium bicarbonate, sodium bisulphate, sodium carbonate, sodium citrate, sodium fumarate, sodium gluconate, sodium hexametaphosphate, sodium hydroxide, sodium lactate, sodium phosphate, dibasic, sodium phosphate, monobasic, sodium phosphate, tribasic, sodium potassium hexametaphosphate, sodium potassium tartrate, sodium potassium tripolyphosphate, sodium pyrophosphate, tetrabasic, sodium tripolyphosphate, sulphuric acid, sulphurous acid, tartaric acid, and a combination thereof.

The formulation may further include a preservative. In particular examples, the preservative includes sodium benzoate, however the preservative is not particularly limited. The preservative includes, but is not limited to, acetic acid, ascorbic acid, calcium ascorbate, erythorbic acid, iso-ascorbic acid, potassium nitrate, potassium nitrite, sodium ascorbate, sodium erythorbate, sodium iso-ascorbate, sodium nitrate, sodium nitrite, wood smoke, benzoic acid, calcium sorbate, Carnobacterium divergens M35, Carnobacterium maltaromaticum CB1, ethyl lauroyl arginate, 4-hexylresorcinol, Leuconostoc carnosum 4010, long-chain glycolipids from Dacryopinax spathularia MUCL 53181, methyl-p-hydroxybenzoate (methylparaben), modified vinegar (a liquid or spray-dried mixture containing acetic acid and one or more of potassium acetate, potassium diacetate, sodium acetate or sodium diacetate, prepared by adding potassium bicarbonate or sodium bicarbonate, potassium carbonate or sodium carbonate, or potassium hydroxide or sodium hydroxide to vinegar), nisin, potassium acetate, potassium benzoate, potassium bisulphite, potassium diacetate, potassium lactate, potassium metabisulphite, potassium sorbate, propionic acid, propyl-p-hydroxybenzoate (propylparaben), sodium acetate, sodium benzoate, sodium bisulphite, sodium diacetate, sodium lactate, sodium metabisulphite, sodium propionate, sodium salt of methyl-p-hydroxybenzoic acid, sodium salt of propyl-p-hydroxybenzoic acid, sodium sorbate, sodium sulphite, sodium dithionite, sorbic acid, sulphurous acid, calcium propionate, calcium sorbate, dimethyl dicarbonate, long-chain glycolipids from Dacryopinax spathularia MUCL 53181, natamycin, potassium sorbate, propionic acid, sodium diacetate, sodium propionate, sodium sorbate, sorbic acid, ascorbic acid, ascorbyl palmitate, ascorbyl stearate, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), citric acid, citric acid esters of mono- and diglycerides, L-cysteine, L-cysteine hydrochloride, Gum Guaiacum, lecithin, lecithin citrate, monoglyceride citrate, monoisopropyl citrate, propyl gallate, sodium metabisulphite, tartaric acid, tertiary butyl hydroquinone, tocopherols (α-tocopherol; tocopherols concentrate, mixed), and combinations thereof.

The formulation may further include a vehicle or carrier medium. The carrier medium may include but is not limited to xylitol, erythritol, inulin, mannitol, salts, and kaolin clay minerals.

In one, non-limiting example, the formulation is manufactured by vacuum drying the extract. 5 g of the powdered extract is dissolved in USP (United States Pharmacopeia) grade ethanol (1000 mL) or another suitable solvent. The extract solution is spray or otherwise impregnated to the carrier medium. The extract-impregnated material is re-extracted and tested to calculate the dose of the psychoactive compound. The extract-impregnated material is mixed with other additives such as a buffering agent, preservative, flavouring agent, spray, solvent, solubilizer, and combinations thereof. The mixture is packed into cellulose capsules for ingestion by a subject.

Table 1 shows the composition of the formulation according to one embodiment.

TABLE 1

| Raw | Qty | | Composition | Tolerance |
|---|---|---|---|---|
| material | (g) | Function | (wt/wt %) | (%) |
| Psilocybe | 1.00 | Medicinal Ingredient | 0.01 | ±5 |
| extract | | | | |
| Xylitol | 1000.00 | Inactive vehicle or | 97.0 | ±5 |
| | | media | | |
| Na-citrate | q.s. | Buffering agent | 0.9 | ±1 |
| Sorbic acid | q.s. | Preservative | 1.00 | ±2 |
| Flavours | q.s. | Taste enhancer | 1.00 | ±2 |
| Ethanol | 1000.00 | Spray agent and | | |
| | | solubilizer | | |

Table 2 shows the composition of the formulation according to another embodiment.

TABLE 2

| Raw | Qty | | Composition | Tolerance |
|---|---|---|---|---|
| material | (g) | Function | (wt/wt %) | (%) |
| Psilocybe | 1.00 | Medicinal Ingredient | 0.01 | ±5 |
| extract | | | | |
| Inulin | 1000.00 | Inactive vehicle or | 97.0 | ±5 |
| | | media | | |
| Na-citrate | q.s. | Buffering agent | 0.9 | ±1 |
| Sorbic acid | q.s. | Preservative | 1.00 | ±2 |
| Flavours | q.s. | Taste enhancer | 1.00 | ±2 |
| Ethanol | 1000.00 | Spray agent and | | |
| | | solubilizer | | |

Table 3 shows the composition of the formulation according to another embodiment.

TABLE 3

| Raw | Qty | | Composition | Tolerance |
|---|---|---|---|---|
| material | (g) | Function | (wt/wt %) | (%) |
| Psilocybe | 1.00 | Medicinal Ingredient | 0.01 | ±5 |
| extract | | | | |
| Erythritol | 1000.00 | Inactive vehicle or | 97.0 | ±5 |
| | | media | | |
| Na-citrate | q.s. | Buffering agent | 0.9 | ±1 |
| Sorbic acid | q.s. | Preservative | 1.00 | ±2 |
| Flavours | q.s. | Taste enhancer | 1.00 | ±2 |
| Ethanol | 1000.00 | Spray agent and | | |
| | | solubilizer | | |

Table 4 shows the composition of the formulation according to another embodiment.

TABLE 4

| Raw | Qty | | Composition | Tolerance |
|---|---|---|---|---|
| material | (g) | Function | (wt/wt %) | (%) |
| Psilocybe | 1.00 | Medicinal Ingredient | 0.01 | ±5 |
| extract | | | | |
| Mannitol | 1000.00 | Inactive vehicle or | 97.0 | ±5 |
| | | media | | |
| Na-citrate | q.s. | Buffering agent | 0.9 | ±1 |
| Sorbic acid | q.s. | Preservative | 1.00 | ±2 |
| Flavours | q.s. | Taste enhancer | 1.00 | ±2 |
| Ethanol | 1000.00 | Spray agent and | | |
| | | solubilizer | | |

It will now be apparent to a person of skill in the art that the present specification affords certain advantages over the prior art. Current research into psychoactive compounds, including psilocybin and psilocin, rely on synthetic compounds because natural extraction is not commercially viable. The present disclosure provides an extraction method that produces a standardized extract with higher yields of psychoactive active compounds.

CO₂ extraction is a clean and safe solvent to produce a standardized extract for the future production of pharmaceutical products. CO₂ leaves no residue in the extract and does not generate a hazardous liquid waste stream. CO₂ is an effective extraction solvent at pressures lower than high pressure water extraction (the supercritical pressures of CO₂ and water are 7.4 MPa and 22 MPa, respectively).

Due to its acidity, CO₂ was thought to be unsuitable for the extraction of psychoactive compounds which are susceptible to degradation in low pH environments. Nonetheless, and as demonstrated herein, CO₂ extraction can produce yields of psychoactive compounds that are as high or higher than conventional extraction techniques. In particular, the CO₂ extraction methods disclosed herein limit oxidation of psychoactive compounds such as psilocin. When oxidized, psilocin is converted to psilocybin, an inactive form of psilocin that cannot be used by the body. Limiting the oxidation of psilocin in an extract using the methods described herein provides a faster-acting formulation. Furthermore, the methods disclosed herein lead to higher and more consistent yields than conventional techniques such as chloroform, water, or ultrasonic extraction.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

The disclosure will now be explained by way of example.

### EXAMPLE

### 1. Overview

The example herein describes a protocol for validating the low-oxygen environment extraction process for psilocin. Data is generated under target low-oxygen conditions and atmospheric conditions.

Psilocin is extracted from the dried, ground mushrooms using carbon dioxide under pressurized conditions, according to method 300 as described above. In the low-oxygen conditions, the extract is deposited into a collection vessel that has been purged with nitrogen gas and contains the antioxidant α-tocopherol. For the atmospheric condition, the extract is deposited into a collection vessel that is open to the laboratory space and contains no preservative.

The concentration of psilocin in the resulting extracts is determined by HPLC at Keystone Labs, Edmonton, Alberta, Canada using an Agilent® 12690 Infinity II analytical HPLC (Agilent: Santa Clara, California).

### 2. Materials and Methods

Data is collected as per this protocol for a minimum of three consecutive commercial scale GMP (good manufacturing practices) runs for each collection condition: low-oxygen and atmospheric.

The fungal biomass comprises aerial parts of Psilocybe cubensis (MyFungi™ plc Calgary, Canada). The fungal biomass is cleaned, freeze-dried targeting moisture levels of 0.1% wt./wt. and ground into 0.4 mm-1.2 mm particles using the Robot-Coupe Blixer 4 (Robot-Coupe: Torrence, California).

The average size of the fungal biomass particles is verified using ASTM (American Society for Testing and Materials) qualified test sieves (ASTM E11-09 compliant), such as model V8SF from the Gilson Company, Inc. (Lewis Center, Ohio). The particle size reduction may include the QA/QC (quality assurance and quality control) size reduction standard of a D₅₀ equal to 400 μm-1200 μm, measured using the ASTM qualified test sieves. D₅₀ is the median particle diameter or median particle size of the fungal biomass.

The dried, ground biomass is stored until extraction.

The fungal biomass is packed into a commercially available, extraction pressure vessel (such as the Superfast 2×9 from Thar Process (Pittsburgh, PA)) to a density of 100-600 kg/m³. Between 1 and 5 kg of biomass is packed into the extraction vessel.

Ethanol or methanol is added to the fungal biomass at a concentration of 5% to 10% (v/v).

Supercritical carbon dioxide is pumped into the extraction vessel at a pressure greater than 7.4 MPa up to 35 MPa in combination with temperatures greater than 304.1 K up to 360.0 K. The flow rate of CO₂ is 1-10 kg per hour. The duration of the contact between biomass and carbon dioxide may be between one minute and 24 hours.

The extraction vessel is rapidly depressurized to atmospheric pressure. The extract is rinsed from the extraction vessel using a solvent and collected into a collection vessel under either low-oxygen conditions (described in section 2.1) or atmospheric conditions (described in section 2.2)

The solvent is evaporated from the extract using a commercially available rotary evaporation system such as the IKA RV10 Digital Pro™ (Cole-Parmer: Quebec, Quebec).

### 2.1 Low-Oxygen Conditions

The procedure for extraction under low-oxygen conditions requires the collection vessel to be purged with nitrogen and have an aliquot of α-tocopherol added to it prior to collection of the extract.

Into the extraction collection container, add an aliquot of α-tocopherol equal to about 0.3% (wt/wt) of the expected mass of extract collected.

A nitrogen purge is conducted as a displacement purge wherein a controlled flow of nitrogen enters the collection vessel through an inlet port and exit the collection vessel via an outlet port. The flow of nitrogen gas is maintained until the volume of nitrogen that has passed through the collection vessel is equivalent to three (3) times the volume of the collection vessel, at which point the outlet port is closed, and the inlet port is closed.

Samples (2×100 μL) is removed from the outlet port of the collection vessel following completion of the extraction. The collection vessel must be in a nitrogen-purged environment (i.e., nitrogen purged glove box) prior to opening and sampling.

### 2.2 Atmospheric Conditions

The procedure for extraction under atmospheric conditions requires the extraction collection container to be used empty (i.e., no α-tocopherol) and to have a headspace containing gas that is from the general laboratory environment.

During sample collection, the outlet and inlet ports of the collection container are open.

Samples (2×100 μL) is removed from the outlet port of the collection container following completion of the extraction. The container must be in the general laboratory environment.

### 2.3 Sample Analysis

All samples removed during production for in-process testing as per this Protocol is held frozen before shipping to Keystone Labs. The psilocin content of the low-oxygen extracts and atmospheric extracts is determined by Keystone Labs by HPLC method.

The data shows that the psilocin yield is higher when the extract is collected in low-oxygen conditions as compared with extracts that are collected under atmospheric conditions, supporting the use of the low-oxygen extraction process.

What is claimed is:

1. A method of extracting a psychoactive compound from fungal biomass, the method comprising the steps of:
(a) contacting the fungal biomass with pressurized carbon dioxide to dissolve an extract of the fungal biomass, the extract including at least one psychoactive compound;
(b) depressurizing the carbon dioxide to separate the extract from the carbon dioxide; and
(c) conveying the extract into a collection vessel.

2. The method of claim 1 wherein the pressurized carbon dioxide is supercritical.

3. The method of claim 1 wherein the at least one psychoactive compound includes a plurality of psychoactive compounds.

4. The method of claim 1 wherein the collection vessel contains a preservative to inhibit oxidation of the at least one psychoactive compound.

5. The method of claim 4 wherein the preservative is nitrogen gas.

6. The method of claim 5 further comprising the step of purging the collection vessel with the nitrogen gas prior to collecting the extract.

7. The method of claim 4 wherein the preservative is α-tocopherol.

8. The method of claim 1 wherein the fungal biomass includes Psilocybe sp.

9. The method of claim 8 wherein the at least one psychoactive compound comprises psilocin.

10. The method of claim 9 wherein the at least one psychoactive compound further comprises at least one of psilocybin, aeruginascin, and a β-carboline.

11. The method of claim 1 further comprising the step of contacting the fungal biomass with a co-solvent.

12. The method of claim 11 wherein the co-solvent is ethanol.

13. The method of claim 11 wherein the co-solvent is methanol.

14. The method of claim 1 further comprising the step of reducing an average particle diameter of the fungal biomass prior to contacting the fungal biomass with the pressurized carbon dioxide.

15. The method of claim 14, wherein after reducing the average particle diameter of the fungal biomass, the average particle diameter of the fungal biomass is about 400 μm to about 1200 μm.

16. The method of claim 1 further comprising drying the fungal biomass prior to contacting the fungal biomass with the pressurized carbon dioxide.

17. The method of claim 16 wherein the dried fungal biomass includes less than 1 wt % water.

18. An extract of Psilocybe sp. comprising the plurality of psychoactive compounds, produced according to the methods of claim 3.

19. A formulation comprising an extract of Psilocybe sp., the extract comprising a plurality of psychoactive compounds including at least one of psilocin, psilocybin, aeruginascin, and a β-carboline.

20. The formulation of claim 19, wherein the extract is obtained according to the methods of claims 1 to 16.

21. The formulation of claim 19 further comprising a carrier medium.

22. The formulation of claim 21 wherein the carrier medium is selected from a group consisting of xylitol, erythritol, inulin, mannitol, salts, kaolin clay minerals, and combinations thereof.

23. The formulation of claim 19 further comprising a buffering agent.

24. The formulation of claim 19 further comprising a flavouring agent.

25. The formulation of claim 19 further comprising a preservative.

26. The formulation of claim 19 further comprising a solvent.

27. The formulation of claim 26 wherein the solvent comprises ethanol.

28. Use of the extract of claim 18 comprising a plurality of psychoactive compounds for the treatment of a mental illness selected from a group consisting of depression, anxiety, obsessive compulsive disorder (OCD), psychosis, post-traumatic stress disorder (PTSD), and addiction.

29. Use of the extract of claim 18 comprising a plurality of psychoactive compounds for the treatment of migraines.

30. Use of the extract of claim 18 comprising a plurality of psychoactive compounds for the treatment of cluster headaches.

31. Use of the formulation according to any one of claims 19 to 27 comprising a plurality of psychoactive compounds for the treatment of a mental illness selected from a group consisting of anxiety, obsessive compulsive disorder (OCD), psychosis, post-traumatic stress disorder (PTSD), disordered eating, and addiction.

32. Use of the formulation according to any one of claims 19 to 27 comprising a plurality of psychoactive compounds for the treatment of migraines.

33. Use of the formulation according to any one of claims 19 to 27 comprising a plurality of psychoactive compounds for the treatment of cluster headaches.

34. Use of the formulation according to any one of claims 19 to 27 comprising a plurality of psychoactive compounds for the treatment of chronic pain.
